(19) United States
(12) Patent Application Publication
(10) Pub. No.: US 2005/0079540 A1
(43) Pub. Date: Apr. 14, 2005

(54) Method for conducting solid phase synthesis of molecule libraries using combinatorial sealing matrices

(76) Inventor: Andre Bernard, Tubingen (DE)

Correspondence Address: NATH & ASSOCIATES, 1030 15th STREET, NW, 6TH FLOOR, WASHINGTON, DC, 20005, US

(21) Appl. No.: 10/475,800

(22) PCT Filed: Apr. 26, 2002
(86) PCT No.: PCT/EP02/04668

(30) Foreign Application Priority Data
Apr. 26, 2001 (DE) 101-21-571.1

(51) Int. Cl. G01N 33/53; G01N 33/543

(52) U.S. Cl. 435/7.1; 436/518

(57) ABSTRACT

The invention relates to a method for producing molecule libraries at predetermined locations on a substrate surface by means of sequential chemical reactions involving the use of combinatorial sealing matrices. The topological structures applied to the sealing matrices cover individual areas of the substrate surface in a defined order thereby preventing different partial areas of the substrate surface from undergoing chemical conversions. Several sealing matrices having different topological relief structures can be used in a number of reaction cycles for the synthesis of molecule libraries consisting of complex chemical compounds. The sealing matrices are made of an elastic material such as polydimethylsiloxane. The synthesis involves the use of simple and highly optimized standard methods and standard chemicals of the solid phase synthesis. The reaction rate is accelerated by carrying out the reaction steps in a microfluidic flow-through system. The inventive method can be used for easily and rapidly producing molecule libraries in the microarray format.

### DESCRIPTION

The present invention relates to a method for producing molecule libraries on a substrate surface by means of sequential chemical reactions.

Complex chemical compounds for analytical purposes are prepared using, as a standard, solid-phase synthesis of DNA oligonucleotides, peptides or other macromolecules, which usually takes place on polystyrene beads or other polymeric carriers.

Thus, for example, the method according to Merrifield (Merrifield, Science 10 232: 341-347 (1986)) is employed as standard in the solid-phase synthesis of peptides and proteins. This method involves synthesizing polypeptides by covalently linking amino acids whose amino groups are protected step by step to the end of a growing polypeptide chain. After coupling the new amino acid to the chain, the protective group on the terminal α-amino group is removed and the next synthetic cycle takes place.

The traditional method for preparing DNA on solid carriers uses phosphoamidite chemistry on polystyrene beads. For this purpose, a protected nucleotide is coupled in each cycle to the growing end of an oligonucleotide chain, the protective groups are removed and a new coupling cycle is started. The reaction conditions and process runs of this method are optimized in such a way that it is possible to obtain yields of from 98% to 99.5% per reaction cycle. For individual steps of this method, the reader is referred to the following review: Beaucage, Iyer, Tetrahedron 48: 2223-2311 (1992).

The traditional methods prove to be too slow and ineffective when preparing a large number of complex chemical compound [sic]. Only a few polymers per day can be prepared using these methods.

A known method for producing a larger number of substances is the synthesis on microtiter plates in the ELISA format. The disadvantage of the traditional methods of solid-phase synthesis in the microtiter-plate format is the necessity of a multiplicity of pipetting and aspirating steps, because reagents are spatially separated due to compartmentation of the well-containing plate or by means of perforated masks as top parts (WO 98/36827). This method requires, for example, up to 80 individual steps for synthesis of a 20-mer oligonucleotide. Moreover, the reaction on the surface takes place with mass-transport limitation, resulting in slower reaction kinetics.

For many applications such as drug screening, gene expression analysis or genetic diagnostics, the number of molecules to be tested is so high that the microtiter-plate format is no longer sufficient for effective and economical synthesis. For problems of this kind, spatially resolved supports without well, referred to as arrays which represent molecule libraries on a solid support are used. The limiting factor in terms of cost and time for mass production of molecule arrays is the application of molecules to metal, glass, membrane or plastic surfaces which needs to be reproduced many times. In principle, the following two techniques of this method are known: 1) an in-situ synthesis of array molecules from monomers by means of photochemically or electrostatically mediated reactions in situ directly on a support (U.S. Pat. No. 5,405,783); 2) applying ready-made macromolecules either in drop form (spotting) by means of printing pin (U.S. Pat. No. 6,101,946), micropipettes (U.S. Pat. No. 5,601,980) or ink jet printers (U.S. Pat. No. 5,927,547). For the current state of the art, in particular for the use of microarrays in DNA and protein analysis, the reader is referred to the following specialist review article: Nature Genetics, Vol. 21, No. 1 supplement (1999).

The known methods for preparing molecule arrays have the disadvantage of requiring elaborate apparatus and are therefore expensive. Furthermore, optical lithographic methods use special light-reactive protective group chemistry which entails low chemical product yields and undesired secondary reactions.

It is the object of the present invention to provide a method for simple and cost-effective production of molecule libraries in the microarray format, which has higher precision and reproducibility.

To achieve this object, the invention primarily proposes a method with the features as defined in claim 1. Developments of the invention are subject matters of the remaining dependent and independent claims 2 to 28, the wording thereof as well as the wording of the abstract are incorporated by reference into this specification.

The method of the invention avoids several of the abovementioned limitations at once and removes the shortcomings of the conventional methods by simple and effective means. This is achieved by transferring the highly optimized and simple standard methods and standard chemicals of the traditional solid-phase synthesis to the microarray format, with location selectivity and the order of the chemical reactions being obtained using combinatorial sealing matrices.

The invention describes a method for space-resolved and selective synthesis of complex chemical compounds at predefined sites on a substrate surface whose entirety forms a dot matrix, a circular, helical, strip-shaped, linear or other geometrical arrangement of reaction sites.

Prior to each reaction cycle, all substrate surface areas to be protected from the particular chemical reaction are covered by means of a topologically structured sealing matrix and thus excluded from the subsequent reaction. All the other, not covered sites of the substrate remain freely accessible to the reaction chemicals.

According to the invention, complex chemical compounds are synthesized at predefined sites on the substrate surface, which can be activated before the start of the reaction. The location selectivity of activation or passivation is made possible by different methods known to the skilled worker, such as, for example, microcontact printing, photolithography or light-selective chemistry. Alternatively, the use of sealing matrices of the invention may ensure location selectivity. For this purpose, the areas on which synthesis is to take place are covered with a sealing matrix and the remaining area is contacted with passivating substances. It is also possible to cover the areas on which no synthesis is to take place and to activate complementary areas subsequently.

The areas designed for the synthesis of macromolecules may be activated by way of example, but not by way of limitation, by location-selective silanization with ω-30 functionalized ethoxy, methoxy or chlorosilanes. It is also possible to use on gold- or silver-coated substrates self assembling monolayers of thiols or disulfides.

Alternatively, an entirely activated surface area may be passivated location-selectively. For this purpose, the areas on which no synthesis is to take place are coated, for example, by microcontact printing with polymers, quenchers or proteins suitable therefore. Thus it is possible, for example, to activate the entire area first by aminosilanization and subsequent coating with N-hydroxysuccinimidyl ester. After covering the fields designed for the synthesis of macromolecules with a sealing matrix, the remaining surface area is passivated with terminal amines, for example by means of ethanolamine or polyethylene glycol.

The structures applied to the sealing matrix and the distances between neighboring structures may have very small dimensions of down to less than 1 μm. The topological structures may be elevations, for example, and form a relief on the matrix surface. The elevations may preferably extend over the full area and accordingly rest completely on the substrate surface when the method of the invention is carried out. Other designs are possible, and the elevation may be formed by an edging, for example. Here, the sealing matrix seals via these edge regions of the elevations when it is placed on the substrate surface.

The basic requirements for the use of this method is a conformal contact with the substrate surface. Moreover, the structural elements must not sag and not be distorted too much when contacting the substrate surface. The structures are characterized here by the aspect ratio (ratio of the height of the structures to their lateral dimensions in periodic structures) and the filling factor (ratio of contact area to total area of said structures) (see also: Delamarche et al., Advanced Materials 9: 741-746 (1997); Bietsch and Michel, J. Appl. Phys. 88: 4310-4318 (2000)). An aspect ratio of from 1:5 to 5:1, in some cases from 1:20 to 20:1 when using suitable materials, is particularly recommendable. The aspect ratio should be at least 5%-10%.

The shortest distance between elevations on the sealing matrix equals the distance between individual neighboring substrate surface areas on which the synthesis is to be carried out. These areas are preferably slightly smaller in size than the cross section area of the elevations on the combinatorial sealing matrices so that, when a matrix makes contact with the substrate surface, the matrix structures project beyond the borders of the areas to be sealed by a few μm.

The activated substrate surface which is in parts hermetically covered by said matrix structures is contacted with the first solution comprising the first reaction chemicals, and the reaction is started. Possible examples of a reaction are a covalent linking reaction, chemical or enzymic modification, coupling, cleavage, hydrolysis, noncovalent bonding and other reactions. The first reaction initiated takes place on the activated substrate surface areas which are not covered by matrix structures. After the first reaction step and, where appropriate, subsequent washing, the first sealing matrix is separated from the substrate surface and a second sealing matrix is put on which again covers individual areas on the substrate surface and leaves uncovered the sites complementary thereto for the second chemical reaction. The substrate surface which has been partly sealed in this way is contacted with a second reaction solution and the second reaction step is initiated. Another sealing matrix and another reaction solution are used for the third reaction step. The number of reaction steps, the reactants and the shape of the topological sealing matrix may be varied as desired. This method may be used to synthesize in combinatorial steps any chemical compounds on a substrate surface.

The synthesis of macromolecules with the aid of topological sealing matrices may be advantageously optimized via the design of the array to be synthesized and also by determining the reaction sequence. The order of reaction steps may be chosen, for example, so as for a chemical reaction to take place on as many areas as possible of the substrate surface in a single step. This enables the number of steps required for the synthesis of all desired molecules to be reduced to a minimum. Moreover, the arrangement on the substrate surface of the chemical compounds to be synthesized may be fixed in such a way that it is possible to contact a plurality of neighboring areas on the substrate surface in a reaction with the same reaction solution. In this case, sealing matrices may be used whose topological structures cover or leave uncovered a plurality of areas at once. This enables larger structures to be generated on the sealing matrix which are easier to prepare and more resistant to wear and tear. Known computer-aided combinatorial methods may be used to calculate an arrangement of the molecules on the substrate, which is optimum for synthesis, to design the sealing matrices and to calculate an optimal reaction step order.

The following demands are made on materials for preparing combinatorial sealing matrices: precise structurability, sealing contact with a suitable substrate (conformal contact), chemical and physical resistance to reaction conditions. Furthermore, in the case of small structures, geometrical alignment which is characterized by accurate positionability and relative position equalization must be ensured. By way of preference but not by way of limitation, the material used for matrix preparation is an elastomer, for example polydimethylsiloxane (PDMS). PDMS is a synthetic material which can be cast into any shapes and cured (Delamarche et al., Advanced Materials 9: 741-746 (1997)). It is particularly suitable as material for preparing sealing matrices, since, owing to its physical properties, it is capable of fitting to the substrate surface with the highest accuracy and thus ensuring even contact and sufficient sealing.

When using strong bases or halogenated hydrocarbons such as, for example, chlorinated solvents in the reaction, it is possible to use harder but chemically inert materials for matrix preparation. In this case, it is possible to construct a hybrid structure made of a hard and chemically resistant core and soft contact faces made of elastic material, in order to ensure sealing with the substrate surface. Examples of hard material which may be used are glass, silicon, gold, silver, nickel or other metals and various plastics. The contact faces of the topological structures may comprise an elastomer such as PDMS or other siloxanes, silicones, gum-like polymers, polyurethanes and other moldable elastic thermoplasts. In addition, chemical modifications may increase the chemical resistance of the sealing matrix and there may be, for example, an increase in the degree of crosslinking of the polymer, glazing of the outer layer of the sealing matrix, surface treatment by applying a thin protective layer composed of a protective polymer or a metal, or other suitable chemical modifications.

Structured sealing matrices are prepared by defining master structures, for example in the form of a silicon wafer or as structured glass by means of classical photolithography, and using said master structures as casting molds for the liquid prepolymer. After curing, the elastic polymer can be removed in the desired three-dimensional form. For the preparation of structures from PDMS, the reader is referred to the following review article: Xia and Whitesides, Angew. Chem. Int. Ed. 37: 550-575 (1998). PDMS is transparent for wavelengths down to the lower UV range. According to the invention, this may be utilized in order to ensure, for example, optical control of matrix positioning relative to the substrate (alignment). Furthermore, particular light-sensitive reactions, for example for coupling molecular components, may be controlled by light.

The method described may be used to prepare from monomers such as, for example, nucleotides, amino acids, sugar units and from other molecular components oligo- and polymers such as DNA, RNA, aptamers and their derivatives such as PNA or thioRNA, peptides, proteins, complex carbohydrates and other chemical compounds.

The method described of solid-phase synthesis of molecule libraries in the array format is suitable both for preparing microarrays of molecules with desired defined compositions and for a controlled combinatorial synthesis of any possible macromolecules from a predetermined number of components. The former [sic] is applied in parallel analysis of biological samples. Thus it is possible to use the arrays prepared according to the method described, for example, in the analysis of gene expression, in genetic diagnostics, in biological and pharmaceutical research, in the determination of genetically engineered organisms in the food industry, etc.

In the controlled combinatorial synthesis, the complete molecular diversity may be generated, starting from predefined reactants. Known computer-aided combinatorial methods may be used, for example, for calculating the reaction order and the particular matrix topologies for preparing any possible kind of molecules. The combinatorial molecule libraries generated in this way may be removed from the substrate surface and assayed in a solution for particular activities. Alternatively, screening may be carried out using the molecules coupled to the solid substrate. In this case, the composition of individual molecules in question can be readily determined, since position information of any synthesized molecules is known due to the fixed reaction order and matrix topologies. The combinatorial molecule libraries generated according to the method described may be used, for example, in the search for new potential active compounds in pharmaceutical research (drug discovery) or in the search for new agrochemicals for agriculture.

Due to the combination of traditional solid-phase synthesis with the highly parallel microarray format, the method of the invention has a number of advantages compared to known methods. Firstly, simple and proven standard techniques and already optimized protocols of the traditional methods can be adopted. Secondly, the reaction rates are significantly increased compared to the traditional methods, since the chemical reactions take place in a microfluidic flow-through system formed by topological microstructures of the sealing matrices. Thirdly, the volumes of the reagents used may be minimized down to nanoliter ranges. Fourthly, the use of standard methods makes possible a substantially simpler operation without elaborate apparatus, compared to known microarray preparation methods. Fifthly, the use of the microarray format avoids very many pipetting and washing steps of traditional solid-phase synthesis so that a multiplicity of different chemical compounds are synthesized in parallel. The invention thus makes possible a high parallelization of the process by relatively simple and cost-effective means. Using the method described, it is possible to prepare a large number of different macromolecules in relatively few steps.

Further advantages, features and possible applications of the invention are described below on the basis of the exemplary embodiments with reference to the drawings in which:

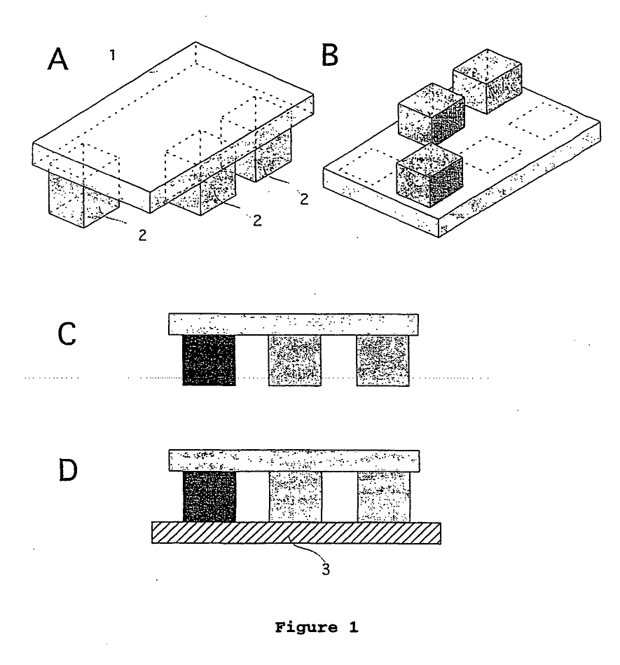

FIG. 1: shows a diagrammatic representation in three views of a sealing matrix for the synthesis of a 2×3 molecule array,

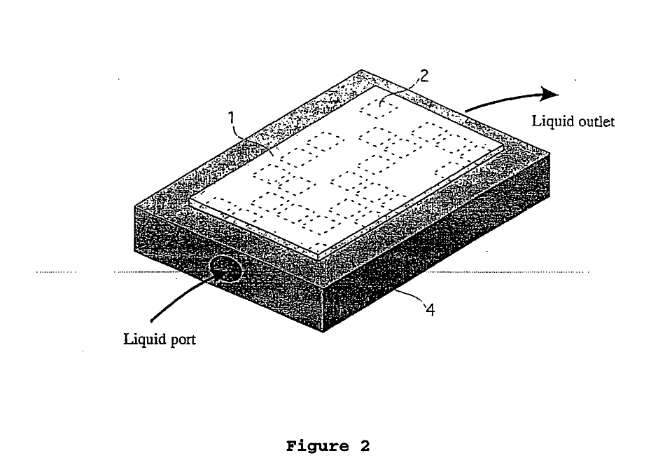

FIG. 2: shows a cassette for carrying out solid-phase synthesis with the aid of combinatorial sealing matrices,

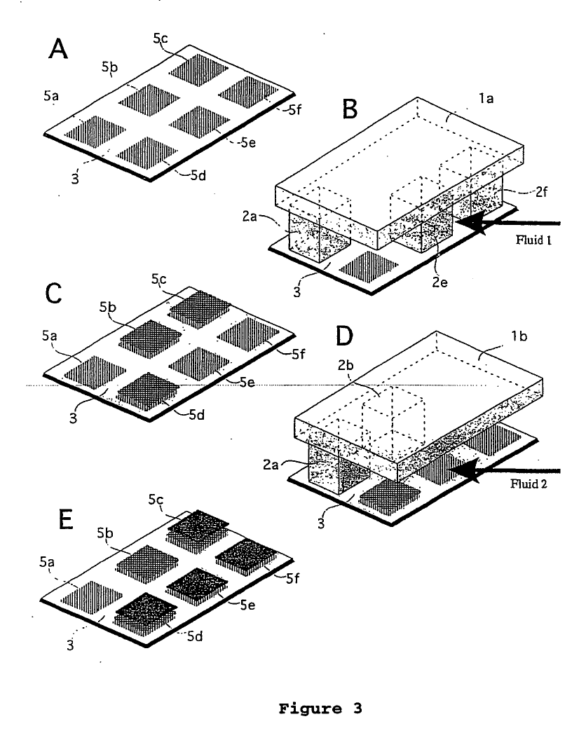

FIG. 3: shows an application example of the synthesis of a 2×3 molecule array on a substrate surface,

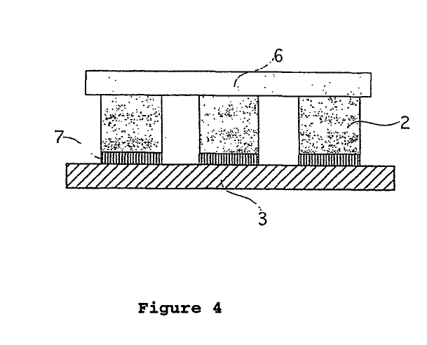

FIG. 4: shows an alternative design of a sealing matrix in the form of a hybrid structure,

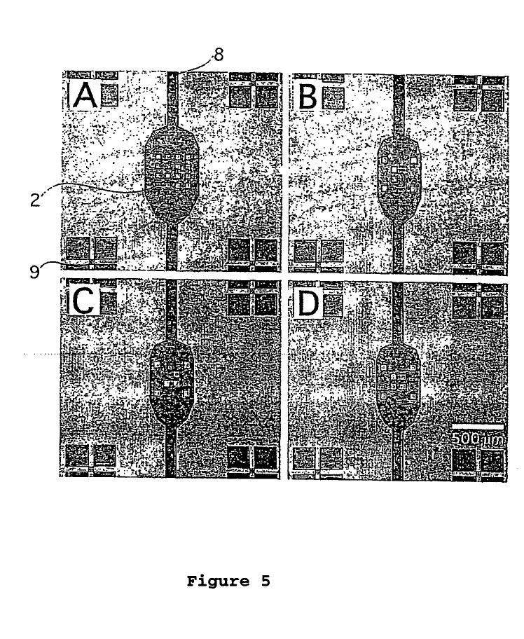

FIG. 5: shows a light-microscopic image of exemplary combinatorial sealing matrices.

### EXAMPLES

FIG. 1 depicts in a diagrammatical and simplified manner three views (FIG. 1A to 1C) an exemplary sealing matrix for preparation of a 2×3 array. Elevations 2 are present at three sites on the depicted sealing matrix 1. When using the depicted sealing matrix in a solid-phase synthesis, the chemical reactions proceed only at the activated sites on the substrate which are opposite the three free areas during contact with a substrate surface. No chemical conversion takes place at the sites of the substrate 3 which come into contact with the elevations (FIG. 1D), since the reaction solutions are prevented from wetting the covered area at these sites.

FIG. 2 depicts a cassette 4 in which solid-phase synthesis by means of combinatorial sealing matrices can take place. In the figure shown, the cassette comprises as an example a sealing matrix 1 with elevations 2 (indicated by dashed lines) for preparation of a 5×7 array. The cassette serves to accurately position the sealing matrices on the substrate which is likewise placed in the cassette. Furthermore, the cassette has two nozzles for introducing and discharging reaction solutions. Cassette, sealing matrix and substrate together form a flow-through system for reaction solutions and for washing reagents. When using sealing matrices with very many and very small structures of less than 50 μm, the frictional forces in the narrow channels between the elevations may become so large that an active pumping force is required which may be achieved, for example, by means of a hydrodynamically or peristaltically generated overpressure or by means of reduced pressure. The transport of liquids by means of capillary forces may likewise be utilized advantageously. For this purpose, a large part of the side walls which form the flow-through system must have a hydrophilic coating when using hydrophilic reaction solutions. When the matrix material used is PDMS, the matrix surface may be hydrophilized, for example, by means of oxygen plasma or by means of a chemical treatment.

FIG. 3 depicts an application example in the form of the synthesis of a 2×3 array of macromolecules from monomers via simple coupling reactions on a substrate surface. The example depicted is a sequence of two reaction steps. A) The substrate surface 3 comprises six activated fields 5a to 5f (highlighted in the figure by hatching) on which the coupling reactions can take place. These fields are slightly smaller than the cross section area of the elevations on the combinatorial sealing matrices and are used for coupling the monomers. It is recommended to design the activated fields so much smaller than the covering elevated areas that the positioning inaccuracy resulting from putting on matrices is still small enough for the activated area to be completely covered by the matrix structures. If, for example, the activated fields have an edge length of 50 μm, then it would be possible to chose for the covering elevated area, for example, an edge length of 80 μm, with half the difference of 15 μm representing the error tolerance of a positioning inaccuracy. Areas between the activated fields are passivated, for example, by using inert surface groups in order to prevent unwanted coupling of the molecules thereto. B) The first monomer should couple to the fields 5b, 5c and 5d of the substrate 3. For this purpose, the first coupling reaction uses a sealing matrix la whose elevations 2a, 2e and 2f seal three complementary fields 5a, 5e and 5f and thus remove them from contact with the reaction solution. The reaction takes place in a cassette, as depicted in FIG. 2. After the first sealing matrix has covered the corresponding fields, the substrate surface is flooded via the inlet port with the liquid or gaseous reaction chemicals comprising the first monomer (arrow). Due to the small dimensions of the matrix structures, the flow is laminar, with a minimal path length for diffusion of the reactants, resulting in accelerated reaction kinetics. C) The monomers are coupled to the free fields 5b, 5c and 5d which have not been sealed (indicated by checkered rectangles). D) In the next step, the second monomer is to be coupled to the fields 5c, 5d, 5e and 5f. For this purpose, a second sealing matrix 1b with two elevations 2a and 2b on the complementary areas which cover, in the second coupling reaction, the fields 5a and 5b is used. The substrate surface 3 is then contacted with another solution comprising the second monomer (arrow). E) After flow-through of the second reaction solution, the second monomer remains coupled to the fields 5c, 5d, 5e and 5f (indicated by dark-gray rectangles). In this case, the second monomer is coupled directly to the activated substrate surface on the fields 5e and 5f and to the previously applied first monomer on the fields 5c and 5d. These two reaction steps already result in three types of molecules: comprising only the first monomer (field 5b), comprising only the second monomer (fields 5e and 5f), and an oligomer comprising the first and the second monomer (fields 5c and 5d). The field 5a remains unaltered after these two reaction cycles. In the next reaction steps, any number of further monomers may be coupled using further topological sealing matrices which may have different or else identical shapes so that the desired oligomers are synthesized at the predefined sites.

FIG. 4 depicts an alternative sealing matrix design which may be used under extreme chemical reaction conditions during sythesis, such as, for example, very high temperatures or the use of strong bases or halogenated hydrocarbons. In this case, it is possible to construct a hybrid structure made of a hard and chemically inert core and a soft elastic contact face. Examples of hard material which may be used are glass, silicon, gold, silver, nickel or other metals and various plastics. The contact faces of the topological structures may comprise elastomers such as PDMS or other siloxanes, silicones, gum-like polymers, polyurethanes and other shapable elastic thermoplasts.

In this case, the main part of the sealing matrix 6 is constructed from the harder material and the elastomer forms a thin layer 7 on the proximal areas of the elevations 2 and thus mediates contact to the substrate 3. In this way, the sealing elastomer remains substantially isolated from the aggressive solutions and reaction conditions.

FIG. 5 depicts a light-microscopic image of exemplary combinatorial sealing matrices, displaying the front view of four different sealing matrices. Elevations 2 of which the image shows the proximal areas are present on the surface of the matrices. The construction of the sealing matrices depicted comprises, in addition to differently arranged elevations, a flow cell 8 through which the reaction solutions flow and adjusting aids 9 which enable the matrices to be precisely positioned on the substrate. The sealing matrix depicted in FIG. 5A, which has sixteen elevations arranged in the form of a dot matrix, may be used, for example, for preparing the substrate surface for the subsequent synthetic steps, for example by activating all fields designed for the coupling of molecular components and passivating the complementary areas. The contact face of the elevations of this preparatory sealing matrix corresponds to the area of activated fields on the substrate surface. The sealing matrices 5B to 5D used in the subsequent synthetic steps have elevations with larger areas than the first preparatory sealing matrix 5A, in order to cover the positioning inaccuracy. The present invention is not limited to the afore-described exemplary embodiments. Rather, skilled workers can achieve a multiplicity of possible variations which are thus included within the scope of the present invention.

1. A method for producing geometrically arranged molecule libraries comprising chemical compounds on a substrate surface, which method is characterized by the following steps:
preparing at least one sealing matrix whose relief-like topological structures ensure a sealing contact with said substrate surface at predefined sites;
contacting said sealing matrix with said substrate surface;
conducting a chemical reaction on the substrate surface areas not covered by said sealing matrix;
separating said sealing matrix from said substrate surface.

2. The method as claimed in claim 1, characterized in that two or more different or identical sealing matrices are used for conducting two or more chemical reactions on the substrate surface.

3. The method as claimed in claim 1, characterized in that the molecule library synthesized on the substrate surface forms an array of different groups of chemical compounds whose composition and location are known.

4. The method as claimed in claim 1, characterized in that the topological structures on the sealing matrices and the chemical compounds synthesized at the predefined sites of the substrate surface are arranged in the form of a dot matrix, a circular, helical, strip-shaped, linear or other geometrical structure.

5. The method as claimed in claim 1, characterized in that the molecule library synthesized on the substrate surface can be used for conducting parallel binding reactions.

6. The method as claimed in claim 1, characterized in that the chemical reaction is covalent linking, chemical or enzymic modification, coupling, cleavage, hydrolysis, noncovalent bonding or another chemical reaction.

7. The method as claimed in claim 1, characterized in that the sealing matrix comprises, at least partially, an elastic material, preferably, polydimethylsiloxane.

8. The method as claimed claim 1, characterized in that different parts of the sealing matrix comprise different materials.

9. The method as claimed claim 1, characterized in that the sealing matrix is made of a material transparent for at least one particular wavelength and thus enables photochemical reactions or near-field optical processes to be carried out via location-selective light conduction.

10. The method as claimed in claim 8, characterized in that the sealing matrix transparent for a particular wavelength enables relative positioning of said sealing matrix on the substrate surface to be controlled via location-selective light conduction.

11. The method as claimed in claim 1, characterized in that the sealing matrix has an electrically conducting surface, in particular for controlling electrochemical reactions.

12. The method as claimed in claim 1, characterized in that a topological structure on the sealing matrix covers a plurality of areas designed for the synthesis of chemical compounds.

13. The method as claimed in claim 1, characterized in that individual areas on the substrate surface, designed for the synthesis of chemical compounds, have a spatial dimension of preferably less than 10 μm, in particular less than 2 μm.

14. The method as claimed in claim 1, characterized in that the substrate surface has activated, geometrically arranged areas on which chemical compounds are synthesized.

15. The method as claimed in claim 14, characterized in that the areas designed for the synthesis of chemical compounds are activated by location-selective silanization with ω-functionalized ethoxy- or methoxy-or chlorosilanes or by applying functionalized thiols or disulfides to noble metal films.

16. The method as claimed in claim 1, characterized in that the contact faces of the topological structures on the sealing matrix are larger than the substrate surface areas designed for the synthesis of chemical compounds.

17. The method as claimed in claim 1, characterized in that the surface areas outside the areas designed for the synthesis of chemical compounds are passivated.

18. The method as claimed in claim 17, characterized in that the areas on which no chemical compounds are to be synthesized are passivated by coating with polymers, quenchers or proteins or by location-selectively deactivating or removing active groups.

19. The method as claimed in claim 1, characterized in that the chemical compounds synthesized on the substrate surface are DNA, RNA, aptamers or their, in particular nuclease-resistant, derivatives such as PNA or thioRNA.

20. The method as claimed in claim 1, characterized in that the chemical compounds synthesized on the substrate surface are peptides, proteins or their derivatives.

21. The method as claimed claim 1, characterized in that the chemical compounds synthesized on the substrate surface are carbohydrates.

22. The method as claimed claim 1, characterized in that the chemical compounds synthesized on the substrate surface are dendrimers or other organic or inorganic macromolecules.

23. An apparatus for preparing the sealing matrices and molecule libraries on a substrate surface according to claim 1, characterized in that individual steps are carried out semi-automatically or automatically.

24. A kit comprising the essential substances for producing molecule libraries according to any of the methods as defined in claim 1.

25. A kit comprising the essential substances for carrying out binding assays on molecule libraries according to any of the methods as defined in claim 1.

26. A sealing matrix having relief-like topological structures, in particular for carrying out the methods as claimed in claim 1.

27. The sealing matrix as claimed in claim 26, characterized by at least one feature of of the following:
(a) that the topological structures on the sealing matrices and the chemical compounds synthesized at the predefined sites of the substrate surface are arranged in the form of a dot matrix, a circular, helical, strip-shaped, linear or other geometrical structure;
(b) the sealing matrix comprises, at least partially, an elastic material, preferably, polydimethylsiloxane;
©) different parts of the sealing matrix comprise different materials;
(d) the sealing matrix is made of a material transparent for at least one particular wavelength and thus enables photochemical reactions or near-field optical processes to be carried out via location-selective light conduction;
(e) the sealing matrix transparent for a particular wavelength enables relative positioning of said sealing matrix on the substrate surface to be controlled via location-selective light conduction;
(f) the sealing matrix has an electrically conducting surface, in particular for controlling electrochemical reactions;
(g) a topological structure on the sealing matrix covers a plurality of areas designed for the synthesis of chemical compounds;
(h) the contact faces of the topological structures on the sealing matrix are larger than the substrate surface areas designed for the synthesis of chemical compounds.

28. A device for carrying out the method as claimed in claim 1, preferably in the form of a cassette, characterized in that it has at least one sealing matrix or is designed for received such a sealing matrix.
